(19) United States
(12) Patent Application Publication
(10) Pub. No.: US 2026/0337648 A1
(43) Pub. Date: Oct. 29, 2026

(54) SYNTHESIS OF FLAVOR MODULATORS

(71) Applicant: GIVAUDAN SA, VERNIER (CH)

(72) Inventors: Zhenhua RUAN, Cincinnati, OH (US); Marco De Jonge, Naarden (NL); Emelie Verhoek, Naarden (NL)

(21) Appl. No.: 19/109,675

(22) PCT Filed: Sep. 8, 2023
(86) PCT No.: PCT/EP2023/074689

§ 371 Date: Mar. 7, 2025

Related U.S. Application Data
(60) Provisional application No. 63/374,957, filed on Sep. 8, 2022.

(51) Int. Cl. C12P 17/16; A23L 27/40

(52) U.S. Cl. CPC C12P 17/165; A23L 27/45

(57) ABSTRACT

The invention relates to a method for preparing a compound of formula (III) or an edible salt thereof, specifically to an enzyme-catalysed method. Such compounds are preferably flavor modulation compounds.

### FIELD

The invention relates to a method for preparing a compound of formula (III) or an edible salt thereof, specifically to an enzyme-catalysed method. Such compounds are preferably flavor modulation compounds.

### BACKGROUND

The synthesis of compounds of formula (III) often involves harsh reaction conditions, low materials and energy efficiencies, and a low yield [1-3].

There is thus a continuing need in the art for efficient synthesis of compounds of formula (III) involving mild reaction conditions, increased materials and energy efficiencies, and increased yields.

### DESCRIPTION OF THE INVENTION

In an aspect, the invention provides a method for preparing a compound of formula (III) or an edible salt thereof, comprising the reaction between carboxylic acid derivative (I) and amine (II) in a solvent, wherein the reaction is catalysed by an enzyme:

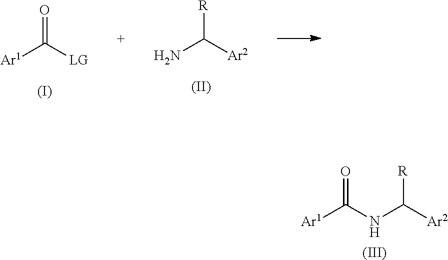

wherein each of Ar¹ and Ar² comprise a heteroaromatic moiety, wherein LG is a leaving group or OH, and wherein R is H or COOH. Such a method may be called a method of or according to the invention herein. Unless explicitly mentioned otherwise, any method, or feature thereof, mentioned herein refers to a method according to the invention.

### Compound and Reactants

A heteroaromatic moiety is an aromatic ring, which may be substituted, comprising at least one endocyclic heteroatom (i.e. an atom different from carbon or hydrogen). Preferably, the heteroatom is nitrogen, oxygen or sulphur, more preferably nitrogen. Examples of heteroaromatic moieties are pyrrole, furan, thiophene, indole, benzofuran, benzothiophene, imidazole, ozazole, thiazole, pyrazole, isoxazole, isothiazole, pyridine, pyrilium, thiapyrilium, quinoline, isoquinoline, carbazole and purine, or any suitable derivates thereof.

An edible salt is readily understood by the skilled person and includes those salts typically employed in the food and beverage industry including chlorides, sulphates, phosphates, gluconates, sodium, citrates, carbonates, acetates and lactates.

Preferably, at least one heteroaromatic moiety comprised in Ar¹ and Ar² comprises a nitrogen atom. Preferably, the combined number of nitrogen atoms comprised in Ar¹ and Ar² is from 1 up to 8, from 1 up to 7, from 1 up to 6, from 1 up to 5, from 1 up to 4, is from 2 up to 8, from 2 up to 7, from 2 up to 6, from 2 up to 5, from 2 up to 4, is from 3 up to 8, from 3 up to 7, from 3 up to 6, from 3 up to 5, from 3 up to 4.

Preferably, at least one of Ar¹ and Ar² comprises an imidazolyl, an indolyl, a pyrimidinyl or a thiazolyl, preferably 1H-4-imidazolyl, 1H-5-indolyl, pyrimidin-2-yl or 1,3-thiazol-2-yl.

Preferably, Ar¹ comprises a heteroaromatic moiety comprising a nitrogen atom. Preferably, the number of nitrogen atoms comprised in Ar¹ is from 1 up to 4, from 1 up to 3, from 1 up to 2, from 2 up to 4, from 2 up to 3. Preferably, the number of nitrogen atoms comprised in Ar¹ is 1, 2, 3, 4, 5 or 6.

Preferably, Ar¹ comprises an imidazolyl, an indolyl, a pyrimidinyl or a thiazolyl, preferably 1H-4-imidazolyl, 1H-5-indolyl, pyrimidin-2-yl or 1,3-thiazol-2-yl.

Preferably, the carboxylic acid derivative (I) can be represented by formula (I-link), wherein Ar^{1R} is a heteroaromatic moiety and L₁ is a linker.

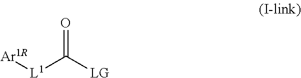

Preferably, Ar^{1R} comprises a heteroaromatic moiety comprising a nitrogen atom. Preferably, the number of nitrogen atoms comprised in Ar^{1R} is from 1 up to 4, from 1 up to 3, from 1 up to 2, from 2 up to 4, from 2 up to 3. Preferably, the number of nitrogen atoms comprised in Ar^{1R} is 1, 2, 3, 4, 5 or 6.

Preferably, Ar^{1R} is an imidazolyl, an indolyl, a pyrimidinyl or a thiazolyl, preferably 1H-4-imidazolyl, 1H-5-indolyl, pyrimidin-2-yl or 1,3-thiazol-2-yl.

Preferably, L₁ is a C₁₋₃ alkylene or C₁₋₃ alkenylene. A C₁₋₃ alkylene is an saturated carbohydrate diradical comprising 1, 2 or 3 carbon atoms. A C₁₋₃ alkenylene is an singly unsaturated carbohydrate diradical comprising 1, 2 or 3 carbon atoms. Preferably, L¹ is methylene (—CH₂—), ethylene (—CH₂—CH₂—), trimethylene (—CH₂—CH₂—CH₂—), —CH(CH₃)—CH₂—, ethenylene (—CH═CH—), propenylene (—C(CH₃)═CH—), wherein each H may independently be substituted by —NH₂, —OH or a halogen, preferably by —NH₂. Preferably, L¹ is ethenylene or 1-amino-ethylene.

Preferably, Ar¹ is 2-(1H-4-imidazolyl)-ethenyl, 1H-5-indolyl, 2-(1H-5-imidazolyl)-ethenyl, 1-amino-2-(1H-4-imidazolyl)-ethyl, (1,3-thiazol-2-yl)-ethenyl, 2,3-dihydro-1H-indol-2-yl or 2-(pyrimidin-2-yl) ethenyl.

Preferably, the carboxylic acid derivative (I) can be represented by formula (IV), which may be called urocanic acid (LG=OH) or an urocanyl ester (LG=OR′):

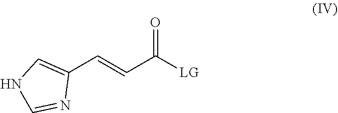

Preferably, Ar² comprises a heteroaromatic moiety comprising a nitrogen atom. Preferably, the number of nitrogen atoms comprised in Ar² is from 1 up to 4, from 1 up to 3, from 1 up to 2, from 2 up to 4, from 2 up to 3. Preferably, the number of nitrogen atoms comprised in Ar² is 1, 2, 3, 4, 5 or 6. Preferably, Ar² comprises imidazolyl, indolyl, pyrimidinyl or thiazolyl, preferably 1H-4-imidazolyl, 1H-5-indolyl, pyrimidin-2-yl or 1,3-thiazol-2-yl.

Preferably, the amine (II) can be represented by formula (II-link), wherein Ar^{2R} is a heteroaromatic moiety and L₂ is a linker:

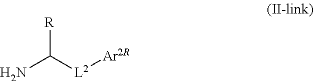

More preferably, the amine (II) can be represented by formula (II-H-link), wherein Ar^{2R} is a heteroaromatic moiety and L₂ is a linker:

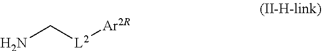

Preferably, Ar^{2R} comprises a heteroaromatic moiety comprising a nitrogen atom. Preferably, the number of nitrogen atoms comprised in Ar^{2R} is from 1 up to 4, from 1 up to 3, from 1 up to 2, from 2 up to 4, from 2 up to 3. Preferably, the number of nitrogen atoms comprised in Ar^{2R} is 1, 2, 3, 4, 5 or 6.

Preferably, Ar^{2R} is an imidazolyl, an indolyl, a pyrimidinyl or a thiazolyl, preferably 1H-4-imidazolyl, 1H-5-indolyl, pyrimidin-2-yl or 1,3-thiazol-2-yl.

Preferably, L² is a C₁₋₃ alkylene or C₁₋₃ alkenylene. Preferably, L² is methylene (—CH₂—), ethylene (—CH₂—CH₂—), trimethylene (—CH₂—CH₂—CH₂—), —CH(CH₃)—CH₂—, ethenylene (—CH═CH—), propenylene (—C(CH₃)═CH—), or —C(CH₃)═CH—, wherein each H may independently be substituted by —NH₂, —OH or a halogen, preferably by —NH₂. Preferably, L² is methylene.

Preferably, Ar² is (1H-imidazol-4-yl)-methyl, (1H-3-indol-3-yl)-methyl, (pyridin-4-yl)methyl, (pyridin-2-yl)methyl or 2-(pyrimidin-2-yl)methyl.

Preferably, the amine (II) can be represented by formula (V), which may be called histamine:

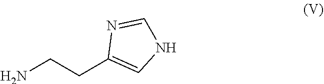

Preferably, Ar¹ and Ar² each independently comprise a heteroaromatic moiety comprising a nitrogen atom. Preferably, the number of nitrogen atoms comprised in each of Ar¹ and Ar² is independently from 1 up to 4, from 1 up to 3, from 1 up to 2, from 2 up to 4, from 2 up to 3. Preferably, the number of nitrogen atoms comprised in each of Ar¹ and Ar² is independently 1, 2, 3, 4, 5 or 6.

Preferably, each of Ar¹ and Ar² independently comprise an imidazolyl, an indolyl, a pyrimidinyl or a thiazolyl, preferably 1H-4-imidazolyl, 1H-5-indolyl, pyrimidin-2-yl or 1,3-thiazol-2-yl.

Preferably, the carboxylic acid derivative (I) can be represented by formula (I-link) and the amine (II) can be represented by formula (II-link), wherein Ar^{1R} and Ar^{2R} are independently heteroaromatic moieties, wherein L¹ and L² are independently linkers.

It is understood that, in the preferable case that the carboxylic acid derivative (I) can be represented by formula (I-link) and the amine (II) can be represented by formula (II-link), the compound can be represented by formula (III-link):

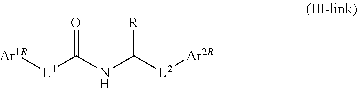

Preferably, the carboxylic acid derivative (I) can be represented by formula (I-link) and the amine (II) can be represented by formula (II-H-link), wherein Ar^{1R} and Ar^{2R} are independently heteroaromatic moieties, wherein L¹ and L² are independently linkers.

It is understood that, in the preferable case that the carboxylic acid derivative (I) can be represented by formula (I-link) and the amine (II) can be represented by formula (II-H-link), the compound can be represented by formula (III-H-link):

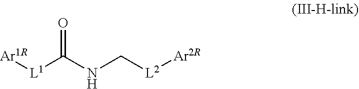

Preferably, Ar^{1R} and Ar^{2R} are independently an imidazolyl, an indolyl, a pyrimidinyl or a thiazolyl, preferably 1H-4-imidazolyl, 1H-5-indolyl, pyrimidin-2-yl or 1,3-thiazol-2-yl.

Preferably, L¹ and L² are independently a C₁₋₃ alkylene or C₁₋₃ alkenylene. Preferably, L¹ and L² are independently methylene (—CH₂—), ethylene (—CH₂—CH₂—), trimethylene (—CH₂—CH₂—CH₂—), —CH(CH₃)—CH₂—, ethenylene (—CH═CH—), propenylene (—C(CH₃)═CH—), or —C(CH₃)═CH—, wherein each H may independently be substituted by —NH₂, —OH or a halogen, preferably by —NH₂. Preferably, L¹ is ethenylene or 1-amino-ethylene, and L² is methylene. More preferably, L¹ is ethenylene and L² is methylene.

Preferably, the carboxylic acid derivative (I) can be represented by formula (IV), i.e. Ar¹ is 2-(1H-4-imidazolyl)-ethenyl, and the amine (II) can be represented by formula (V), i.e. Ar² is (1H-imidazol-4-yl)-methyl, and the compound can thus be represented by formula (VI), which may be called N-urocanylhistamine:

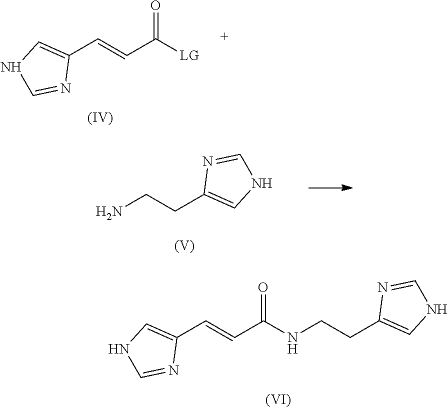

Preferably, the compound of formula (III) can be represented by formula (VI-E). Alternatively, the compound of formula (III) can be represented by formula (VI-Z):

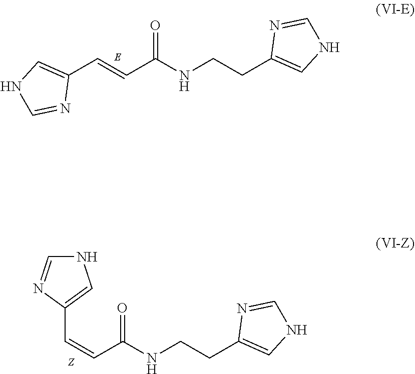 [SMILES: O=C(/C=C/c1c[nH]cn1)NCCc1c[nH]cn1 ; O=C(/C=C\c1c[nH]cn1)NCCc1c[nH]cn1]

Unless explicitly mentioned otherwise, the preferences for certain substituent groups mentioned above may be applied to all Markush formulas in which these substituent group appear. Hence, all preferences for Ar¹ mentioned herein may be applied to formulas (I) and (III). All preferences for LG may be applied to formulas (I), (I-link) and (IV). All preferences for Ar² mentioned herein may be applied to formulas (II) and (III). All preferences for R mentioned herein may be applied to formulas (II), (II-link) and (III). All preferences for Ar^{1R} and L¹ mentioned herein may be applied to formulas (I-link), (III-link) and (III-H-link). All preferences for Ar^{2R} and L² mentioned herein may be applied to formulas (II-link), (II-H-link), (III-link) and (III-H-link).

It is further understood that the formulae and chemical names presented herein are meant to refer to all compounds or moieties which are generally recognized by the skilled person to be represented by those formulae or chemical names. Specifically, unless explicitly mentioned otherwise, and without being limiting, a formula or chemical formula refers to all relevant stereoisomers (diastereoisomers, E/Z isomers, cis/trans isomers, conformers, rotamers, enantiomers, etc.), isotopologues, isotopomers, tautomers, protonated species, (edible) salts etc. as recognized by the skilled person. As an important example, a double bond not part of a ring system may be either E or Z if no specific E/Z-isomer is denoted in a chemical name or if a formula does not contain a letter E or Z next to the double bond. As another example, the structures drawn for the heteroaromatic moieties comprised in Ar¹ and Ar² refer to all tautomers of these moieties.

### Hydrolase-Catalysed Reactions

Preferably, the enzyme used during a method according to the invention is a hydrolase. More preferably, the enzyme is a hydrolase and LG is a leaving group. Most preferably, the enzyme is a hydrolase, LG is a leaving group and the solvent is a non-aqueous solvent, preferably a tertiary alcohol.

A hydrolase is a polypeptide having hydrolase activity, i.e. able to catalyse hydrolyses and (trans) esterifications, preferably in non-aqueous media.

A leaving group has its customary meaning here. In the context of the current invention, a leaving group is able to be displaced easily under the conditions at which the reaction (I)+(II)→(III) is performed. A skilled person is capable of selecting appropriate leaving groups. Preferred leaving groups leave as anions via heterolysis, and can stabilize the additional electron density that results from bond heterolysis. Preferred leaving groups are alkoxy groups (LG=OR′, wherein R is an alkyl group) and halogens (preferably LG=CI, Br or I).

Preferably, LG is OR′, wherein R′ is an alkyl group. More preferably, R′ is a C₁₋₈ alkyl, C₁₋₇ alkyl, C₁₋₆ alkyl, C₁₋₅ alkyl, C₁₋₄ alkyl, C₁₋₃ alkyl, C₂₋₆ alkyl, C₂₋₅ alkyl, C₂₋₄ alkyl, C₂₋₃ alkyl. A C_{x-y} alkyl is a saturated carbohydrate monoradical comprising from x up to y carbon atoms, which may be linear, branched or cyclic. Most preferably, R′ is methyl.

Wherever the enzyme is a hydrolase such as a lipase or an amide synthase, the solvent is preferably a non-aqueous solvent. It should be understood that this does not exclude the presence of water or trace amounts thereof during the reaction between the carboxylic acid derivative (I) and the amine (II), but simply specifies the solvent as not being water.

Preferably, the carboxylic acid derivative (I) and the amine (II) are soluble in the non-aqueous solvent, more preferably expressed by one of the solubility values below, while at the same time allowing the enzyme to catalyse the reaction according to the invention.

Preferably, the solubility of the carboxylic acid derivative (I) in the non-aqueous solvent is at least 0.5, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10, 11, 12, 13, 14, 15, 20, 30, 40, 50, 60, 70, 80, 90, or 100 g/L at a temperature of 20° C. and an a pressure of 1 atm.

Preferably, the solubility of the amine (II) in the non-aqueous solvent is at least 0.5, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10, 11, 12, 13, 14, 15, 20, 30, 40, 50, 60, 70, 80, 90, or 100 g/L at a temperature of 20° C. and at a pressure of 1 atm.

Preferably, the solubility of the carboxylic acid derivative (I) and the solubility of the amine (II) in the non-aqueous solvent is at least 0.5, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10, 11, 12, 13, 14, 15, 20, 30, 40, 50, 60, 70, 80, 90, or 100 g/L at a temperature of 20° C. and an a pressure of 1 atm.

Preferably, the non-aqueous solvent is a tertiary alcohol. Preferably, a tertiary alcohol is 2-methyl-2-butanol, tert-amyl alcohol, tert-butyl alcohol or mixtures thereof. More preferably, a tertiary alcohol is 2-methyl-2-butanol.

Preferably, the enzyme used during a method according to the invention is a lipase (a type of hydrolase). More preferably, the enzyme is a lipase and LG is a leaving group. Most preferably, the enzyme is a lipase, LG is a leaving group and the solvent is a non-aqueous solvent, preferably a tertiary alcohol.

A lipase is a polypeptide, preferably a serine hydrolase, having lipase activity, preferably in non-aqueous media. Their physiological function is the hydrolysis of triglycerides into di- or monoglycerides, fatty acids and glycerol. The lipase may be any type of lipase from the EC 3.1 class. For example, a lipase may be a sn-1-specific diacylglycerol lipase (EC 3.1.1.116), an acylglycerol lipase (EC 3.1.1.23), a galactolipase (EC 3.1.1.26), a triacylglycerol lipase (EC 3.1.1.3), a phospholipase A1 (EC 3.1.1.32), a lipoprotein lipase (EC 3.1.1.34), a phospholipase A2 (EC 3.1.1.4), a lysophospholipase (EC 3.1.1.5), an hormone-sensitive lipase (EC 3.1.1.79). The EC number refers to Enzyme Nomenclature 1992 from NC-IUBMB, Academic Press, San Diego, Calif., including supplements 1-5 published in Eur. J. Biochem., 1994, 223:1-5; Eur. J. Biochem., 1995, 232:1-6; Eur. J. Biochem., 1996, 237:1-5; Eur. J. Biochem., 1997, 250:1-6; and Eur. J. Biochem., 1999, 264:610-650; respectively. The nomenclature is regularly supplemented and updated; see, the World Wide at e.g., Web chem.qmw.ac.uk/iubmb/enzyme/index.html.

A lipase may be obtained by culturing a suitable microorganism and subsequently isolating a lipase expressed by the microorganism. Suitable microorganisms for this purpose belong to the genera Mucor, Aspergillus, Rhizopus, Rhizomucor, Pseudomonas, Candida, Humicola, Thermomyces, Burkholderia ubonensis and Penicillium. Preferred species are Candida antarctica, Thermomyces lanuginosus and Rhizomucor miehei. A lipase that may be obtained by culturing a suitable microorganism and subsequently isolating a lipase expressed by the microorganism, may also be called a lipase from a microbial source herein.

Preferably, a lipase is Candida antarctica lipase-B (Cal-B).

Preferably, the enzyme used during a method according to the invention is a protease (a type of hydrolase). More preferably, the enzyme is a protease and LG is a leaving group. Most preferably, the enzyme is a protease, LG is a leaving group and the solvent is a non-aqueous solvent, preferably a tertiary alcohol.

A protease is a polypeptide having protease activity, preferably in non-aqueous media. Their physiological function is the hydrolysis of proteins or peptides into smaller peptides or single amino acids. The protease may be any type of peptidase from the EC 3.4 class. For example, a protease may be a subtilisin (EC 3.4.21.62).

A protease may be obtained by culturing a suitable microorganism and subsequently isolating a protease expressed by the microorganism. Suitable microorganisms for this purpose belong to the genera Mucor, Aspergillus, Rhizopus, Rhizomucor, Pseudomonas, Candida, Humicola, Thermomyces, Penicillium, Burkholderia and Bacillus. Preferred species are Candida antarctica, Thermomyces lanuginosus, Rhizomucor miehei and Burkholderia ubonensis, more preferably Candida antarctica, Thermomyces lanuginosus, Rhizomucor miehei and Bacillus licheniformis. A protease that may be obtained by culturing a suitable microorganism and subsequently isolating a protease expressed by the microorganism, may also be called a protease obtained from a microbial source herein.

Preferably a lipase or a protease is immobilized (“immobilized lipase” or “immobilized protease”). An immobilized lipase or protease is a lipase or protease which is adsorbed onto a carrier. A carrier is an inert, solid material. An immobilized protease or lipase may be held in suspension in the phase wherein the reaction (I)+(II)→(III) is performed, e.g. via agitation. In the case of an immobilized enzyme, the expression “reaction ( . . . ) in a solvent” refers to the fact that species (I), (II) and (III) react mainly and/or are dissolved in the solvent phase, and does not refer to the fact that the enzyme should be dissolved in the solvent. Without being bound to this theory, the carrier together with the immobilized enzyme forms a separate phase, on whose boundary with the solvent phase the reaction (I)+(II)→(III) is catalysed. Preferably, an immobilized lipase or protease is used in combination with a non-aqueous solvent.

The lipase or protease may be immobilized via any common immobilization technique known to the skilled person. Examples of immobilization techniques are found in J. Mol. Cat. B: Enzymatic 6 (1999) 29-39; Chibata et al. Biocatalysis: Immobilized cells and enzymes, J. Mol. Cat. 37 (1986) 1-24; Sharma et al., Immobilized Biomaterials Techniques and Applications, Angew. Chem. Int. Ed. Engl. 21 (1982) 837-854; Laskin (Ed.), Enzymes and Immobilized Cells in Biotechnology, which are expressly incorporated by reference herein in its entirety. It is generally known that an immobilized enzyme often shows increased resistance to organic solvents as compared to the enzyme in the unbound state. Thus, it can be assumed that once the enzyme is immobilized or stabilized by other methods such as crosslinked enzyme crystals (Altus Biologics, Inc.), higher concentrations of the enzyme may be used. When immobilized on a solid support, for example on a column, the concentration of the enzyme in the reaction mixture could approach 100%. Therefore, various changes, modifications or alternations to the enzyme or the reaction conditions may be made without departing from the spirit or scope of the invention.

Preferably, a carrier is a particulate material having a particle size of 100 to 2000 micrometres. Alternatively, a carrier material may be a membrane.

Preferably, a carrier is a porous material. A porous material preferably has an average pore diameter of at least 20 nanometres.

Preferably, a carrier is a hydrophobic material or am ion-exchange resin.

A hydrophobic material may be selected from polypropylene, polyolefin, polystyrene, polyacrylate (ester), inorganic materials like silicate, silica, glass, zeolites, diatomaceous earth, bentonite, vermiculite, hydrotalcite, agarose, cellulose etc. or combinations thereof. Materials like silica etc. which are normally hydrophilic have to be treated with a suitable compound e.g. a silane to render them hydrophobic.

An ion-exchange resin may be selected from ion-exchange resins based on polystyrene, polyacrylate, phenol-formaldehyde resins and silicas. Ideally, the ion-exchange resin is an anion exchanger, especially a macroporous weak anion exchange resin. Suitable examples include phenol-formaldehyde, polystyrenic, and styrene-DVB resins such as are available under the Trade Marks DUOLITE ES568 and AMBERLYST A21.

Preferably, the lipase is immobilized and obtained from a microbial source. More preferably, the lipase is immobilized and obtained from Candida. Most preferably, the lipase is immobilized and obtained from Candida antarctica.

Preferably, an immobilized lipase is Novozym 435, Lipozyme 435, Lipozyme RM IM, Alcalase CLEA, CalB immo 8806, CalB immo plus.

Novozym 435 (N435) is a commercially available immobilized lipase produced by Novozymes. It is based on immobilization via interfacial activation of lipase B from Candida antarctica on a resin, Lewatit VP OC 1600. This resin is a macroporous support formed by poly(methyl methacrylate) crosslinked with divinylbenzene.

Lipozyme 435 is a commercially available immobilized lipase from Novozymes, which is a food grade (activity >9000 PLU) variant of Novozyme 435, which is a technical grade (activity >10000 PLU).

Lipozyme RM IM is a commercially available immobilized lipase from Novozymes derived from the Rhizomucor miehei fungus, immobilized in phenolic resin.

Alcalase CLEA [4] is an immobilized protease, which is from the company CLEA Technologies.

### Amide Synthase/Synthetase-Catalysed Reactions

Preferably, the enzyme used during a method according to the invention is an amide synthase. More preferably, the enzyme is an amide synthase and LG is OH. Most preferably, the enzyme is an amide synthase, LG is OH and the solvent is an aqueous solvent.

An aqueous solvent may be water, any aqueous buffer, a mixture of water and a water-miscible solvent, or any mixtures thereof.

An amide synthase or amide synthetase is a polypeptide having amide synthase activity, i.e. able to catalyse the formation of an amide bond starting from a carboxylic acid or carboxylate and an amine, preferably in aqueous media. The amide synthase may be any type of amide synthase from the EC 6.3 class. For example, the amide synthase may be a ribosomal peptide synthetase or a nonribosomal amide synthetase. Alternatively, the amide synthetase may be from another EC class. For example, the amide synthetase may use an amide precursor which is biosynthesised by an acid-thiol ligases [5].

An amide synthase may be obtained by culturing a suitable microorganism and subsequently isolating an amide synthase expressed by the microorganism. Suitable microorganisms for this purpose belong to the genera Mucor, Aspergillus, Rhizopus, Rhizomucor, Pseudomonas, Candida, Humicola, Thermomyces, Penicillium, Marinactinospora and Burkholderia. Preferred species are Candida antarctica, Thermomyces lanuginosus, Rhizomucor miehei, Marinactinospora and Burkholderia ubonensis.

Preferably, an amide synthase is Marinactinospora thermotolerans ATP-dependent amide bond synthetase (McbA).

### Other Process Parameters

Preferably, a method according to the invention is performed in the presence of a molecular sieve. A molecular sieve may be a zeolite material with pores of precise and uniform structure and size. This allows them to preferentially adsorb gases and liquids based on molecular size and polarity. Zeolites are naturally existing, highly porous crystalline solids, belonging to the class of chemicals known as aluminosilicates. Preferably, a molecular sieve is a zeolite of one of four types: 3 A, 4 A, 5 A, and 13X. Their chemical formulae are ⅔K₂O₁₃·Na₂₂O·Al₂O₃·2SiO₂·4.5H₂O (3 A), Na₂O·Al₂O₃·2SiO₂·4.5H₂O (4 A), ¾CaO¼Na₂OAl₂O₃·2SiO₂·4.5H₂O (5 A), Na₂O·Al₂O₃·2.45SiO₂·6.0H₂O (13X).

Without being bound to this theory, the presence of a molecular sieve such as 4 A helps to remove water during the reaction (I)+(II)→(III) by adsorption. Since the presence of water may interfere with the reaction (e.g. by hydrolyzing (III) or competing with (II) for binding (I)), this adsorption may increase the yield and the rate of the reaction.

Preferably, a method according to the invention is performed at a temperature from 20° C. up to 100° C., from 20° C. up to 90° C., from 20° C. up to 80° C., from 20° C. up to 70° C., from 20° C. up to 60° C., from 20° C. up to 50° C., from 20° C. up to 40° C., 30° C. up to 100° C., from 30° C. up to 90° C., from 30° C. up to 80° C., from 30° C. up to 70° C., from 30° C. up to 60° C., from 30° C. up to 50° C., from 30° C. up to 40° C., 40° C. up to 100° C., from 40° C. up to 90° C., from 40° C. up to 80° C., from 40° C. up to 70° C., from 40° C. up to 60° C., from 40° C. up to 50° C., 50° C. up to 100° C., from 50° C. up to 90° C., from 50° C. up to 80° C., from 50° C. up to 70° C., from 50° C. up to 60° C. More preferably, a method according to the invention is performed at a temperature from 40° C. up to 80° C. Most preferably, a method according to the invention is performed at a temperature from 50° C. up to 70° C.

Preferably, the ratio between the molar concentrations of (I) and (II) in the solvent is from 1/2.00 up to 2.00, from 1/1.95 up to 1.95, from 1/1.90 up to 1.90, from 1/1.85 up to 1.85, from 1/1.80 up to 1.80, from 1/1.75 up to 1.75, from 1/1.70 up to 1.70, from 1/1.65 up to 1.65, from 1/1.60 up to 1.60, from 1/1.55 up to 1.55, from 1/1.50 up to 1.50, from 1/1.45 up to 1.45, from 1/1.40 up to 1.40, from 1/1.35 up to 1.35, from 1/1.30 up to 1.30, from 1/1.25 up to 1.25, from 1/1.20 up to 1.20, from 1/1.15 up to 1.15, from 1/1.10 up to 1.10, or from 1/1.05 up to 1.05.

Preferably, the ratio between the molar concentrations of (I) and (II) in the solvent is from 0.50 up to 2.0, from 0.55 up to 1.9, from 0.60 up to 1.8, from 0.65 up to 1.7, from 0.70 up to 1.6, from 0.75 up to 1.5, from 0.80 up to 1.4, from 0.85 up to 1.3, or from 0.90 up to 1.2.

Preferably, the molar concentration of (I) in the solvent is from 1 mM up to 100 mM, from 1 mM up to 90 mM, from 1 mM up to 80 mM, from 1 mM up to 70 mM, from 1 mM up to 60 mM, from 1 mM up to 50 mM, from 1 mM up to 40 mM, from 1 mM up to 30 mM, 5 mM up to 100 mM, from 5 mM up to 90 mM, from 5 mM up to 80 mM, from 5 mM up to 70 mM, from 5 mM up to 60 mM, from 5 mM up to 50 mM, from 5 mM up to 40 mM, from 5 mM up to 30 mM, 10 mM up to 100 mM, from 10 mM up to 90 mM, from 10 mM up to 80 mM, from 10 mM up to 70 mM, from 10 mM up to 60 mM, from 10 mM up to 50 mM, from 10 mM up to 40 mM, from 10 mM up to 30 mM, 10 mM up to 100 mM, from 10 mM up to 90 mM, from 10 mM up to 80 mM, from 10 mM up to 70 mM, from 10 mM up to 60 mM, from 10 mM up to 50 mM, from 10 mM up to 40 mM, from 10 mM up to 30 mM.

Preferably, the molar concentration of (II) in the solvent is from 1 mM up to 100 mM, from 1 mM up to 90 mM, from 1 mM up to 80 mM, from 1 mM up to 70 mM, from 1 mM up to 60 mM, from 1 mM up to 50 mM, from 1 mM up to 40 mM, from 1 mM up to 30 mM, 5 mM up to 100 mM, from 5 mM up to 90 mM, from 5 mM up to 80 mM, from 5 mM up to 70 mM, from 5 mM up to 60 mM, from 5 mM up to 50 mM, from 5 mM up to 40 mM, from 5 mM up to 30 mM, 10 mM up to 100 mM, from 10 mM up to 90 mM, from 10 mM up to 80 mM, from 10 mM up to 70 mM, from 10 mM up to 60 mM, from 10 mM up to 50 mM, from 10 mM up to 40 mM, from 10 mM up to 30 mM, 10 mM up to 100 mM, from 10 mM up to 90 mM, from 10 mM up to 80 mM, from 10 mM up to 70 mM, from 10 mM up to 60 mM, from 10 mM up to 50 mM, from 10 mM up to 40 mM, from 10 mM up to 30 mM.

Preferably, the ratio between the molar concentrations of (I) and (II) in the solvent is from 0.5 up to 2.0, preferably wherein the molar concentration of (I) in the solvent is from 1 mM up to 100 mM; or the ratio between the molar concentrations of (I) and (II) in the solvent is from 0.8 up to 1.4, preferably wherein the molar concentration of (I) in the solvent is from 10 mM up to 50 mM; or the ratio between the molar concentrations of (I) and (II) in the solvent is from 0.9 up to 1.2, preferably wherein the molar concentration of (I) in the solvent is from 20 mM up to 30 mM.

Preferably, the concentration of the enzyme in the solvent is from 1 g/L up to 100 g/L, from 1 g/L up to 90 g/L, from 1 g/L up to 80 g/L, from 1 g/L up to 70 g/L, from 1 g/L up to 60 g/L, from 1 g/L up to 50 g/L, 10 g/L up to 100 g/L, from 10 g/L up to 90 g/L, from 10 g/L up to 80 g/L, from 10 g/L up to 70 g/L, from 10 g/L up to 60 g/L, from 10 g/L up to 50 g/L, 20 g/L up to 100 g/L, from 20 g/L up to 90 g/L, from 20 g/L up to 80 g/L, from 20 g/L up to 70 g/L, from 20 g/L up to 60 g/L, from 20 g/L up to 50 g/L, 30 g/L up to 100 g/L, from 30 g/L up to 90 g/L, from 30 g/L up to 80 g/L, from 30 g/L up to 70 g/L, from 30 g/L up to 60 g/L, from 30 g/L up to 50 g/L.

The concentration of an immobilized enzyme in the solvent is defined herein as the mass of the immobilized enzyme (including the mass of the carrier) to the volume of the solution (or suspension) comprising the solvent. In other words, the concentration does not refer to the concentration of dissolved enzymes.

The concentration of a non-immobilized enzyme in the solvent is simply defined herein as the mass of the non-immobilized enzyme to the volume of the solution (or suspension) comprising the solvent.

The method according to any one of the preceding claims, wherein the compound is of formula (VI), wherein the enzyme is an immobilized lipase, wherein LG is OR′, wherein R′ is a C₁₋₄ alkyl, wherein the solvent is a tertiary alcohol, wherein the reaction is performed at a temperature from 20° C. up to 100° C., and wherein the molar concentrations of (I) and (II) in the solvent is from 0.5 up to 2.0, preferably wherein the molar concentration of (I) in the solvent is from 1 mM up to 100 mM.

The method according to any one of the preceding claims, wherein the compound is of formula (VI), wherein the enzyme is an immobilized lipase, wherein LG is OMe, wherein the solvent is a tertiary alcohol, wherein the reaction is performed at a temperature from 20° C. up to 100° C., and wherein the molar concentrations of (I) and (II) in the solvent is from 0.5 up to 2.0, preferably wherein the molar concentration of (I) in the solvent is from 1 mM up to 100 mM.

The method according to any one of the preceding claims, wherein the compound is of formula (VI), wherein the enzyme is an immobilized lipase, wherein LG is OMe, wherein the solvent is a tertiary alcohol, wherein the reaction is performed at a temperature from 40° C. up to 80° C., and wherein the molar concentrations of (I) and (II) in the solvent is from 0.5 up to 2.0, preferably wherein the molar concentration of (I) in the solvent is from 20 mM up to 30 mM.

The method according to any one of the preceding claims, wherein the compound is of formula (VI), wherein the enzyme is an immobilized lipase, wherein LG is OMe, wherein the solvent is 2-methyl-2-butanol, wherein the reaction is performed at a temperature from 40° C. up to 80° C., and wherein the molar concentrations of (I) and (II) in the solvent is from 0.5 up to 2.0, preferably wherein the molar concentration of (I) in the solvent is from 20 mM up to 30 mM.

The method according to any one of the preceding claims, wherein the compound is of formula (VI), wherein the enzyme is an immobilized lipase, wherein LG is OMe, wherein the solvent is a tertiary alcohol, wherein the reaction is performed at a temperature from 40° C. up to 80° C., and wherein the molar concentrations of (I) and (II) in the solvent is from 0.9 up to 1.2, preferably wherein the molar concentration of (I) in the solvent is from 20 mM up to 30 mM.

### Flavor Modulation and Other Aspects

In embodiments, a method according to the invention comprises the isolation of the compound. The isolation may be performed using any common method known to the skilled person. For example, the crude reaction mixture resulting from the enzymatic reaction between (I) and (II) may be filtrated or centrifuged to remove the solids (e.g. the molecular sieve and/or immobilized enzyme), silica flash column chromatography or LC-MS equipped with prep amide column were applied to isolate and purify (III) from the liquid phase of the enzymatic reaction mixture. Finally, any remaining solvent (e.g. water miscible solvent) may be removed by vacuum distillation (e.g. using a Rotovap™) and water may be removed by freeze drying. Further analytics are carried out on the final product using LC-MS or HPLC and compared with authentic standards. More details of this exemplary isolation can be found in Example 1.

In an aspect, the invention provides any compound of formula (III) as described herein, preferably obtainable by any method according to the invention as described herein.

In an aspect, the invention provides a method for preparing a consumer product or flavor composition comprising a compound of formula (III), comprising the step of preparing the compound of formula (III) via a method according to the invention.

Examples of consumer products include a savoury food, a non-savoury food such as dairy, a beverage and confectionery. Examples of a flavor composition includes a savoury flavoring and a sour/acid flavoring. Further examples of consumer products are soups, sauces, stocks, bouillons, cheese products, dressings, seasonings, margarines, noodles and beverages.

Preferably, the consumer product is a food product or a beverage, said consumer product comprising at least 1 ppm, preferably at least 20 ppm, more preferably at least 50 ppm or 70 ppm ppb of one or more compounds of formula (III) and/or edible salts thereof.

Preferably, the consumer product being a food product or a beverage contains at least 0.0001 wt. %, more preferably at least 0.0003 wt. %, even more preferably at least 0.001 wt. %, most preferably at least 0.003 wt. % of the one or more compounds of formula (III). Typically, the aforementioned products will contain the compounds of formula (III) in a concentration of not more than 1 wt. %, preferably of not more than 0.5 wt. %.

Preferably, the consumer product being a food product or a beverage is a low-sodium food product. Without being bound to this theory, the compounds of formula (III) comprised in the low-sodium food product is able to mask the bitter taste of KCl therein.

Preferably, the compound of formula (III) is a flavor modulation compound, i.e. possesses flavor modulating properties. For example, some of the compounds can enhance the salty taste perception, and/or reduce bitter taste and/or enhance umami taste.

In an aspect, the invention provides a method to confer, enhance, improve or modify the flavor properties of a flavor composition or a consumer product, comprising the step of preparing a compound of formula (III) via a method according to the invention and the step of adding the compound of formula (III) to the flavor composition or the consumer product.

Throughout this document the terms “taste” and “flavor” are used interchangeably to describe the sensory impact that is perceived via the mouth, especially the tongue, and the olfactory epithelium in the nasal cavity.

The term “flavor modulating compound” refers to a compound that has no flavor properties as such. However, said substance is capable of altering or complementing or modulating the taste impact of other flavoring substances contained in a flavor composition or in a consumer product, including the salty taste impact, acidic taste impact, bitterness and/or umami taste impact.

For example, the flavor modulating compound does not have any salty taste at all up to levels above 1000 ppm. In combination with salty flavor compounds, for example with NaCl, it can enhance the salty taste perception.

For example, the flavor modulating compound does not have any recognizable taste at a level above 1000 ppm for counteracting or masking bitter taste. In combination with compounds which can have bitter off taste, for example KCl, the bitter taste is reduced.

For example, the flavor modulating compound does not have any recognizable taste at a level above 1000 ppm for counteracting or masking sour taste. In combination with compounds which can have sour off taste, for example NH4Cl, the sour taste is reduced.

For example, the flavor modulating compound does not impart any umami taste at a level above 1000 ppm. In combination with compounds which can impart umami taste, for example MSG, the umami taste is enhanced.

The flavor modulating compounds can be applied advantageously to impart desirable taste attributes to the aforementioned consumer products. In addition, the taste improving substances are capable of modulating the taste impact of other flavor ingredients contained within these same products, thereby improving the overall flavor quality of these products.

The term “flavoring substance” refers to any substance that is capable of imparting a detectable flavor impact, especially at a concentration below 0.1 wt. %, more preferably below 0.01 wt. %. For example, such flavoring substance may be selected from natural flavors, artificial flavors, spices, seasonings, and the like, synthetic flavor oils and flavor aromatics and/or oils, oleoresins, essences, distillates, and extracts derived from plants, leaves, flowers, fruits, and so forth, Generally, any flavor or food additive such as those described in Chemicals Used in Food Processing, publication 1274, pages 63-258, by the National Academy of Sciences, can be used. This publication is incorporated herein by reference.

Flavor modulating compounds can be used in flavor compositions in conjunction with one or more ingredients or excipients conventionally used in flavor compositions beside flavoring substances, for example carrier materials and other auxiliary agents commonly used in the art. Suitable excipients for flavor compositions are well known in the art and include, for example, without limitation, solvents (including water, alcohol, ethanol, oils, fats, vegetable oil, and miglyol), binders, diluents, disintegrating agents, lubricants, flavor agents, coloring agents, preservatives, antioxidants, emulsifiers, stabilisers, flavor-enhancers, anti-caking agents, and the like.

Examples of such carriers or diluents for flavor compositions may be found in for example, “Perfume and Flavour Materials of Natural Origin”, S. Arctander, Ed., Elizabeth, N.J., 1960; in “Perfume and Flavour Chemicals”, S. Arctander, Ed., Vol. I & II, Allured Publishing Corporation, Carol Stream, USA, 1994; in “Flavourings”, E. Ziegler and H. Ziegler (ed.), Wiley-VCH Weinheim, 1998, and “CTFA Cosmetic Ingredient Handbook”, J. M. Nikitakis (ed.), 1st ed., The Cosmetic, Toiletry and Fragrance Association, Inc., Washington, 1988.

Other suitable and desirable ingredients of flavor compositions are described in standard texts, such as “Handbook of Industrial Chemical Additives”, ed. M. and I. Ash, 2nd Ed., (Synapse 2000).

### General Definitions

In this document and in its claims, the verb “to comprise” and its conjugations is used in its non-limiting sense to mean that items following the word are included, but items not specifically mentioned are not excluded. In addition, the verb “to consist” may be replaced by “to consist essentially of” meaning that a product, an assay device respectively a method or a use as defined herein may comprise additional component(s) respectively additional step(s) than the ones specifically identified, said additional component(s), respectively step(s) not altering the unique characteristic of the invention.

In addition, reference to an element by the indefinite article “a” or “an” does not exclude the possibility that more than one of the element is present, unless the context clearly requires that there be one and only one of the elements. The indefinite article “a” or “an” thus usually means “at least one”.

All patent and literature references cited in the present specification are hereby incorporated by reference in their entirety.

### LEGEND TO THE FIGURES

FIG. 1—Enzymatic synthesis kinetics of compound (VI) in 2-methyl-2-butanol with (a) or without (w/o) molecular sieve 4 A (b)

FIG. 2—Molar yield of compound (VI) by enzymatic synthesis with or w/o molecular sieve 4 A

### REFERENCES

1. Brault, G., et al., Short-chain flavor ester synthesis in organic media by an E. coli whole-cell biocatalyst expressing a newly characterized heterologous lipase. PLOS One, 2014. 9(3): p. e91872.
2. Longo, M. A. and M. A. Sanromán, Production of food aroma compounds: microbial and enzymatic methodologies. Food Technology and Biotechnology, 2006. 44(3): p. 335-353.
3. Vandamme, E. J. and W. Soetaert, Bioflavours and fragrances via fermentation and biocatalysis. Journal of Chemical Technology & Biotechnology: International Research in Process, Environmental & Clean Technology, 2002. 77(12): p. 1323-1332.
4. Nuijens, T., et al., Enzymatic C-terminal amidation of amino acids and peptides. Tetrahedron Letters, 2012. 53(29): p. 3777-3779.
5. Petchey, M. R. and G. Grogan, Enzyme-catalysed synthesis of secondary and tertiary amides. Advanced Synthesis & Catalysis, 2019. 361(17): p. 3895-3914.
6. Kokusho, Y., H. Machida, and S. Iwasaki, Studies on alkaline lipase: isolation and identification of lipase producing microorganisms. Agricultural and Biological Chemistry, 1982. 46(5): p. 1159-1164.

### EXAMPLES

The invention is explained in more detail below with a number of examples, which are not to be construed as limiting the scope of the invention. The invention is not limited to the forms of implementation described in the cases given as examples. The invention also extends to each combination of measures as described above, independently from each other.

### Example 1

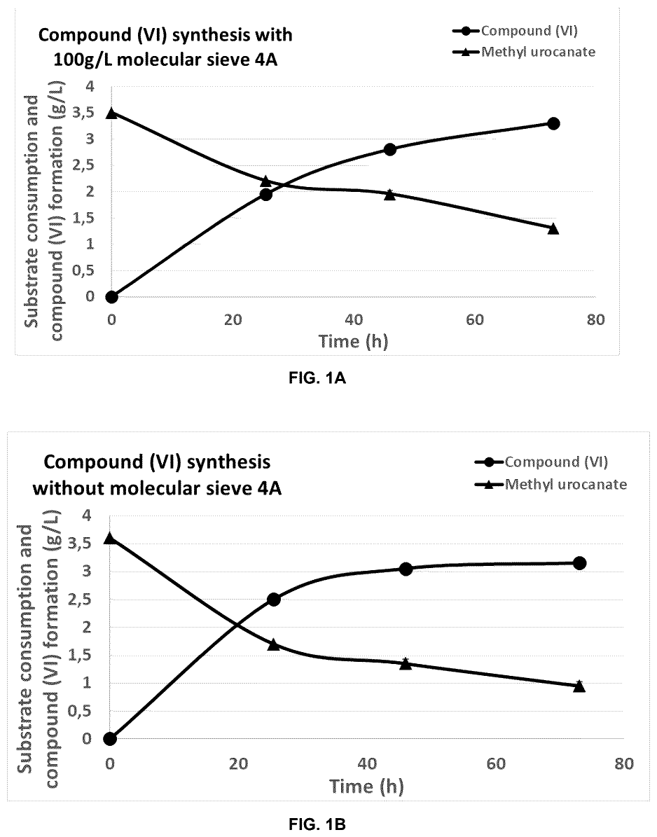

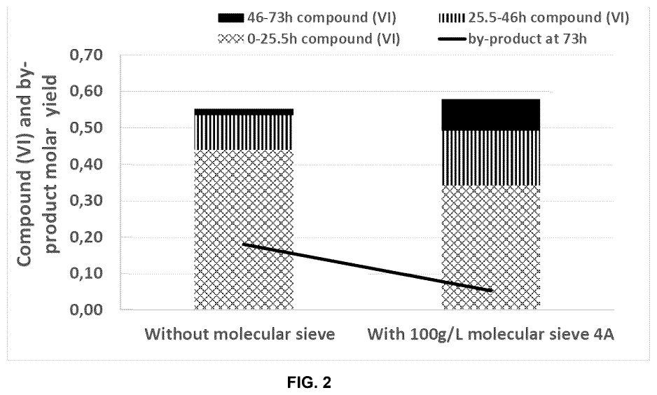

The enzymatic synthesis of compound (VI) was carried out in organic solvent 2-methyl-2-butanol with 25 mM methyl urocanate and 25 mM compound (V) as the starting material as well as 40 g/L Lipozyme 435 (Novozymes) at 60° C. for 73 h, the enzymatic kinetics were shown in FIGS. 1(a) and (b). There was no significant difference of compound (VI) synthesis between (a) and (b). The enzymatic molar conversion yield of compound (VI) was summarized in FIG. 2. There were two treatments, one without the addition of molecular sieve 4 A, while the other one had additional 100 g/L molecular sieve 4 A. Molecular sieve 4 A absorbs excess water and by-product methanol, which are generally undesirable during enzymatic synthesis. According to FIG. 1, the enzymatic reaction with 100 g/L molecular sieve 4 A not only had slightly higher product molar yield (0.58 mol/mol) than that without additional molecular sieve 4 A (0.55 mol/mol), but also lower by-product formation.

The compound (VI) in crude reaction mixture resulting from the enzymatic reaction between methyl urocanate and compound (V) was isolated and purified using either silica flash column chromatography or preparative amide column with LC-MS.

Biotage SNAP Ultra 10 g silica column (Biotage HP-Sphere 25 μm) was used for flash column chromatography. The elution rate was 20 mL/min and mobile phase was the mixture of dichloromethane and methanol (with 0.1% ammonia). The gradient elution of mixed solvent was from 10 to 20% dichloromethane (90 to 80% methanol) for 170 mL, 20 to 30% dichloromethane (80 to 70% methanol) for 170 mL, 30 to 50% dichloromethane (70 to 50% methanol) for 170 mL, and 50 to 70% dichloromethane (50 to 30% methanol) for 170 mL. The glass tubes containing product N-urocanylhistamine were collected and went through roto-evaporation for solvent removal and freeze drying for water removal.

Waters preparative amide column (XBridge BEH Prep OBD Amide 5 μm, 19×250 mm) equipped with single quadrupole LC-MS was used during the separation. Isocratic elution of a solvent mixture of 80% acetonitrile with 0.1% formic acid and 20% water with 0.1% formic acid was used with a flow rate of 16 mL/min. The glass tubes containing product N-urocanylhistamine were collected and went through roto-evaporation for solvent removal and freeze drying for water removal prior to NMR analysis.

### Example 2

The enzymatic synthesis of compound (VI) was carried out in organic solvent 2-methyl-2-butanol with 25 mM methyl urocanate and 25 mM compound (V) as the starting material as well as 40 g/L Lipase TLIM (Novozymes) and 100 g/L molecular sieve 4 A at 60° C. for 72 h. The molar yield of compound (VI) was around 0.018 mol/mol.

### Example 3

The enzymatic synthesis of compound (VI) was carried out in organic solvent 2-methyl-2-butanol with 25 mM methyl urocanate and 25 mM compound (V) as the starting material as well as 40 g/L Lipase TL (Meito sanyo) and 100 g/L molecular sieve 4 A at 50° C. for 72 h. The molar yield of compound (VI) was around 0.14 mol/mol.

### Example 4

The enzymatic synthesis of compound (VI) was carried out in organic solvent 2-methyl-2-butanol with 25 mM methyl urocanate and 25 mM compound (V) as the starting material as well as 40 g/L Lipase QLM (Meito sanyo) and 100 g/L molecular sieve 4 A at 60° C. for 72 h. The molar yield of compound (VI) was around 0.017 mol/mol.

### Example 5

The enzymatic synthesis of compound (VI) was carried out in organic solvent 2-methyl-2-butanol with 25 mM methyl urocanate and 25 mM compound (V) as the starting material as well as 40 g/L CalB immo plus (Purolite) and 100 g/L molecular sieve 4 A at 60° C. for 71 h. The molar yield of compound (VI) was around 0.21 mol/mol.

### Example 6

The enzymatic synthesis of compound (VI) was carried out in organic solvent 2-methyl-2-butanol with 25 mM methyl urocanate and 25 mM compound (V) as the starting material as well as 40 g/L CalB immo 8806 (Purolite) and 100 g/L molecular sieve 4 A at 60° C. for 71 h. The molar yield of compound (VI) was around 0.18 mol/mol.

### Example 7

The enzymatic synthesis of compound (VI) was carried out in organic solvent 2-methyl-2-butanol with 25 mM methyl urocanate and 25 mM compound (V) as the starting material as well as 40 g/L Lipozyme CalB and 100 g/L molecular sieve 4 A at 60° C. for 72 h. The molar yield of compound (VI) was around 0.12 mol/mol.

### Example 8

The enzymatic synthesis of compound (VI) was carried out in organic solvent 2-methyl-2-butanol with 25 mM methyl urocanate and 25 mM compound (V) as the starting material as well as 40 g/L Subtilisin A and 100 g/L molecular sieve 4 A at 60° C. for 72 h. The molar yield of compound (VI) was around 0.21 mol/mol.

### Example 9

The Tables I and II below suitable enzymes that can be used in a method according to the invention, without being limiting.

TABLE I

| Enzyme | | |
|---|---|---|
| product name | Supplier | Description |
| Lipozyme 435 | Novozymes | Immobilized enzyme composed of a Candida antartica B lipase; |
| | | absorbed onto a spherical bead-form carrier based on crossed- |
| | | linked; methacrylic esters. |
| Lipozyme TLIM | Novozymes | A 1,3 specific lipase originating from Thermomyces lanuginosus |
| | | and immobilized on a non-compressible silica gel carrier |
| Lipase TL | Meito sanyo | Extracellular enzyme produced by Pseudomonas Stutzeri that |
| | | has high activities on cholesterol esters |
| Lipase QLM | Meito sanyo | Extracellular enzyme produced by Burkholderia ubonensis that |
| | | shows a wide specificity and high activity towards a variety of |
| | | esters |
| CalB immo 8806 | Purolite | Lipase from Candida Antarctica B Immobilized by adsorption on |
| | | ECR8806M |
| CalB immo plus | Purolite | Lipase from Candida Antarctica B Immobilized by adsorption on |
| | | ECR1030M |
| Lipozyme CalB | Novozymes | Candida antartica B lipase |
| Subtilisin A | Millipore-Sigma (A | Proteaase from Bacillus licheniformis |
| | product of Novozymes) | |

TABLE II

| Enzyme product | Producing | | | | Optimal | Optimum | |
|---|---|---|---|---|---|---|---|
| name | strains | Form | Activity | | pH | temperature | |
| Lipozyme 435 | Candida | Immobilized | 10000 | PLU/g (a) | pH 5-9 | 30-60 | °C |
| | antartica B | | | | | | |
| Lipozyme TLIM | Thermomyces | Immobilized on | 250 | IUN/g (b) | pH 6-8 | 50-75 | °C |
| | lanuginosus | silica gel carrier | | | | | |
| Lipase TL | Pseudomonas | Powder | 50,000 | U/g (c) | pH7-8 | 50 | °C |
| | Stutzeri | | | | | | |
| Lipase QLM | Burkholderia | Powder | 60,000 | U/g (c) | pH 7-9 | 60-70 | °C |
| | ubonensis | | | | | | |
| CalB immo 8806 | Candida | Immobilized by | >10,000 | PLU/g (d) | | | |
| | antartica B | adsorption on | | | | | |
| | | ECR8806M | | | | | |
| CalB immo plus | Candida | Immobilized by | >9,000 | PLU/g (d) | | | |
| | antartica B | adsorption on | | | | | |
| | | ECR1030M | | | | | |
| Lipozyme CalB | Candida | Liquid | 5000 | LU/g (e) | pH 5-9 | 30-60 | °C |
| | antartica B | | | | | | |
| Subtilisin A | Bacillus | Liquid | >2.4 | U/g (f) | pH 6.5- | 60 | °C |
| | licheniformis | | | | 8.5 | | |

(a): PLU=Propyl Laurate Unit, 1 PLU is the amount of enzyme activity which generates 1 μmol of propyl laurate per minute under defined standard conditions.
(b): IUN=Interesterification Unit. 1 PLU is equal to 1 IUN.
(c): assayed by the method of Kokusho et al using olive oil emulsified with polyvinylalcohol as a substrate [6]. 1 U corresponds to the amount of enzyme which liberates 1 μmol fatty acid per min at 37° C.
(d): PLU: one unit corresponds to the synthesis of 1 μmol per minute of propyl laurate from lauric acid and 1-propanol at 60° C.
(e): LU: Lipase Unit, 1 LU is the amount of enzyme activity which liberates 1 μmol of tritratable butyric acid from the substrate glycerol tributyrate per minute under defined standard conditions.
(f): Activity expressed in Anson units.

### Example 11

The enzymatic synthesis of compound (VI) is carried out in organic solvent 4-methyl-2-pentanol (a secondary alcohol) with 25 mM methyl urocanate and 25 mM compound (V) as the starting material as well as 40 g/L Lipozyme 435 (Novozymes) at 60° C. for 72 h. There are two treatments, one without the addition of molecular sieve 4 A, while the other one has additional 100 g/L molecular sieve 4 A. Molecular sieve 4 A absorbs excess water and by-product methanol, which are generally undesirable during enzymatic synthesis.

### Example 12

The amidation reaction is performed with 25 mM urocanic acid and 25 mM compound (V) in aqueous system (pH 7-7.5 buffer solution) with amide synthetase at 37° C. for 24-72 h.

1. A method for preparing a compound of formula (III) or an edible salt thereof, comprising the reaction between carboxylic acid derivative (I) and amine (II) in a solvent, wherein the reaction is catalysed by an enzyme: 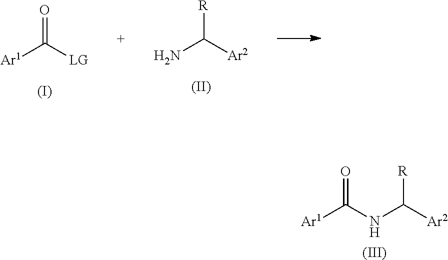
wherein each of Ar¹ and Ar² comprise a heteroaromatic moiety,
wherein LG is a leaving group or OH,
wherein R is H or COOH.

2. The method of claim 1, wherein at least one heteroaromatic moiety comprised in Ar¹ and Ar² comprises a nitrogen atom.

3. The method of claim 1, wherein each of Ar¹ and Ar² independently comprise imidazolyl, indolyl, pyrimidinyl or thiazolyl.

4. The method of claim 1, wherein Ar¹ is 2-(1H-4-imidazolyl)-ethenyl, 1H-5-indolyl, 2-(1H-5-imidazolyl)-ethenyl, 1-amino-2-(1H-4-imidazolyl)-ethyl, (1,3-thiazol-2-yl)-ethenyl, 2,3-dihydro-1H-indol-2-yl or 2-(pyrimidin-2-yl) ethenyl.

5. The method of claim 1, wherein Ar² is (1H-imidazol-4-yl)-methyl, (1H-3-indol-3-yl)-methyl, (pyridin-4-yl)methyl, (pyridin-2-yl)methyl or 2-(pyrimidin-2-yl)methyl.

6. The method of claim 1, wherein Ar¹ is 2-(1H-4-imidazolyl)-ethenyl, wherein Ar² is (1H-imidazol-4-yl)-methyl, wherein the compound is of formula (IV):

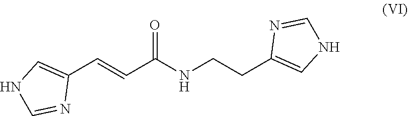 [SMILES: O=C(/C=C/c1c[nH]cn1)NCCc1c[nH]cn1]

7. The method of claim 1, wherein the enzyme is a hydrolase and wherein LG is a leaving group.

8. The method of claim 7, wherein LG is OR′, wherein R′ is a C₁₋₄ alkyl.

9. The method of claim 7, wherein the solvent is a non-aqueous solvent and the hydrolase is a lipase.

10. The method of claim 9, wherein the non-aqueous solvent is a tertiary alcohol.

11. The method of claim 1, wherein the enzyme is an amide synthase, LG is OH and the solvent is an aqueous solvent.

12. The method of claim 1, wherein the reaction is performed in the presence of a molecular sieve.

13. The method of claim 1, wherein the reaction is performed at a temperature from 40° C. up to 80° C.

14. The method of claim 1, wherein the ratio between the molar concentrations of (I) and (II) in the solvent is from 0.9 up to 1.2.

15. The method of claim 1, wherein the concentration of the enzyme in the solvent is from 30 g/L up to 50 g/L.

16. The method of claim 1, wherein the method comprises the isolation of the compound.

17. The method of claim 1, wherein the compound is a flavor modulation compound.

18. The method of claim 10, wherein the tertiary alcohol comprises 2-methyl-2-butanol.
